(19) United States
(12) Patent Application Publication
(10) Pub. No.: US 2007/0203078 A1
(43) Pub. Date: Aug. 30, 2007

(54) METHOD FOR THE TREATMENT AND DIAGNOSIS OF CERTAIN PSYCHIATRIC DISORDERS RELATED TO THE MENSTRUAL CYCLE

(75) Inventors: Istvan Mody, Rancho Palos Verdes, CA (US); Jamie L. Maguire, Los Angeles, CA (US); Brandon M. Stell, Los Angeles, CA (US); Mahsan Rafizadeh, Los Angeles, CA (US)

Correspondence Address: JOHN P. O'BANION;O'BANION & RITCHEY LLP, 400 CAPITOL MALL SUITE 1550, SACRAMENTO, CA, 95814, US

(73) Assignee: THE REGENTS OF THE UNIVERSITY OF CALIFORNIA, Oakland, CA (US)

(21) Appl. No.: 11/623,684

(22) Filed: Jan. 16, 2007

Related U.S. Application Data
(63) Continuation of application No. PCT/US05/26170, filed on Jul. 21, 2005.
(60) Provisional application No. 60/590,659, filed on Jul. 23, 2004.
(60) Provisional application No. 60/620,502, filed on Oct. 19, 2004.

(51) Int. Cl. A61K 31/704; A61K 31/4741; A61K 31/195; A61K 31/56

(52) U.S. Cl. 514/26; 514/292; 514/561; 514/171

(57) ABSTRACT

A method for treating psychiatric disorders associated with the menstrual cycle, such as PMS/PMDD and catamenial epilepsy. Reduced activity of specific subtypes of GABA-A receptors is treated by administering compounds that promote activity of these receptors to restore normal function.

### CROSS-REFERENCE TO RELATED APPLICATIONS

This application claims priority from, and is a 35 U.S.C. §111(a) continuation of, co-pending PCT international application serial number PCT/US2005/026170, filed on Jul. 21, 2005, incorporated herein by reference in its entirety, which claims priority from copending U.S. provisional application Ser. No. 60/590,659, filed on Jul. 23, 2004, incorporated herein by reference in its entirety, and from copending U.S. provisional application Ser. No. 60/620,502, filed on Oct. 19, 2004, incorporated herein by reference in its entirety.

This application is also related to PCT International Publication No. WO 2006/012563 A2, published Feb. 2, 2006, and WO 2006/012563 A3, published Jul. 13, 2006. Each of these publications is incorporated herein by reference in its entirety.

### STATEMENT REGARDING FEDERALLY SPONSORED RESEARCH OR DEVELOPMENT

This invention was made with Government support under Grant No. R37 NS30549, awarded by NIH/NINDS. The Government has certain rights in this invention.

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND OF THE INVENTION

1. Field of the Invention

This invention pertains generally to the treatment of Premenstrual Syndrome (PMS), Late Luteal Phase Dysphoric Disorder (LLPPD) or Premenstrual Dysphoric Disorder (PMDD) and catamenial epilepsy, and more particularly to novel means of modulating the function of specific δ subunit-containing γ-aminobutyric acid type-A receptor (GABAAR) assemblies for treating certain psychiatric disorders and certain psychiatric symptoms including stress, anger, worry, rejection sensitivity and lack of mental or physical energy associated with PMS, LLPD, or PMDD.

2. Incorporation by Reference of Publications

The following publications referenced herein are incorporated by reference herein in their entirety:

Bianchi M T, Macdonald R L (2003) Neurosteroids shift partial agonist activation of GABA(A) receptor channels from low- to high-efficacy gating patterns. J Neurosci 23:10934-10943.

Brambilla P, Perez J, Barale F, Schettini G, Soares J C (2003) GABAergic dysfunction in mood disorders. Mol Psychiatry 8: 721-37, 715.

Brown N, Kerby J, Bonnert T P, Whiting P J, Wafford K A (2002) Pharmacological characterization of a novel cell line expressing human α4β3δ GABA_{A} receptors. Br J Pharmacol 136: 965-974.

Caraiscos V B, Elliott E M, You T, Cheng V Y, Belelli D, Newell J G, Jackson M F, Lambert J J, Rosahl T W, Wafford K A, MacDonald J F, Orser B A (2004) Tonic inhibition in mouse hippocampal CA1 pyramidal neurons is mediated by α5 subunit-containing γ-aminobutyric acid type A receptors. Proc Natl Acad Sci USA 101:3662-3667.

Hevers W, Lüddens H (1998) The diversity of GABA_{A} receptors. Pharmacological and electrophysiological properties of GABA_{A} channel subtypes. Mol Neurobiol 18: 35-86.

Macdonald R L, Olsen R W (1994) GABA_{A} receptor channels. Annu Rev Neurosci 17: 569-602.

McKernan R I V I, Whiting P J (1996) Which GABA_{A}-receptor subtypes really occur in the brain? Trends Neurosci 19: 139-143.

Mody I (2001) Distinguishing between GABA(A) receptors responsible for tonic and phasic conductances. Neurochem Res 26: 907-913.

Nusser Z, Mody I (2002) Selective modulation of tonic and phasic inhibitions in dentate gyrus granule cells. J Neurophysiol 87: 2624-2628.

Saxena N C, Macdonald R L (1996) Properties of putative cerebellar gamma-aminobutyric acid A receptor isoforms. Mo! Pharmacol 49: 567-579.

Semyanov A, Walker M C, Kullmann D M, Silver R A (2004) Tonically active GABA(A) receptors: modulating gain and maintaining the tone. Trends Neurosci 27: 262-269.

Sieghart W, Sperk G (2002) Subunit composition, distribution and function of GABA(A) receptor subtypes. Cun Top Med Chem 2: 795-816.

Stell B M, Brickley S G, Tang C Y, Farrant M, Mody I (2003) Neuroactive steroids reduce neuronal excitability by selectively enhancing tonic inhibition mediated by delta subunit-containing GABAA receptors. Proc Natl Acad Sci USA 100: 14439-14444.

Stoffel-Wagner B (2003) Neurosteroid biosynthesis in the human brain and its clinical implications. Ann N Y Acad Sci 1007: 64-78.

Waliner M, Hanchar H J, Olsen R W (2003) Ethanol enhances α4β3δ and α6β3δ GABA_{A} receptors at low concentrations known to have effects in humans. Proc Natl Acad Sci USA 100: 15218-15223.

Whiting P J (2003) The GABA_{A} receptor gene family: new opportunities for drug development. Curr Opin Drug Discov Devel 6: 648-657.

An example of related work can also be found in U.S. Published Patent Application No. 2004/0024038 A1, published on Feb. 5, 2004 and incorporated herein by reference in its entirety. That application describes the use of a non-steroid compound which acts on the GABA receptor for treatment of disorders relating to reduced neurosteroid activity. The non-steroid compounds may be GABA agonists, GABA uptake inhibitors or enhancers of GABAergic activity.

3. Description of Related Art

Premenstrual Syndrome (PMS) is a term used to describe a variety of physical and emotional changes associated with specific phases of the menstrual cycle. The term Late Luteal Phase Dysphoric Disorder (LLPDD), or the more recent term Premenstrual Dysphonic Disorder (PMDD), is used to describe severe forms of PMS. For the purpose of simplicity, all terms will be referred to as PMS/PMDD. The essential feature of PMS/PMDD is a pattern of clinically significant emotional and behavioral symptoms that occur during the last week of the luteal phase and remit within a few days after the onset of the follicular phase. In most females, these symptoms occur in the week before and remit within a few days after the onset of menses. PMDD is diagnosed only if the symptoms are sufficiently severe to cause marked impairment in social or occupational functioning and have occurred during a majority of menstrual cycles in the past year. The diagnostic criteria rely mostly on subjective reporting of individual conditions rather than on a scientifically defined measure of the condition.

The present approaches for treating PMS/PMDD include diet changes and administration of various compounds, including non-steroidal anti-inflammatory drugs, progesterone, lithium carbonate, thiazide, diuretics, antidepressants, and bromocyptone. Effectiveness of prescribed drugs, however, has often been uncertain or limited.

In addition to the symptoms of PMS/PMDD, cyclic hormonal changes contribute to catamenial epilepsy, which is characterized by seizures occurring around specific points in the menstrual cycle.

### BRIEF SUMMARY OF THE INVENTION

In general terms, the invention comprises a method for treating psychiatric disorders associated with the menstrual cycle, such as PMS/PMDD and catamenial epilepsy. Beneficially, reduced activity of specific subtypes of GABA-A receptors is treated by administering compounds that promote activity of these receptors to restore normal function.

In one embodiment of the invention, a method of regulating expression of δ subunit-containing GABA-A receptors comprises administering a nontoxic dose of a compound targeting enhancement of neurosteroid-sensitive tonic GABAergic inhibition.

In one embodiment, the compound comprises a GABA-A receptor agonist. In one embodiment, the GABA-A receptor agonist comprises 4,5,6,7-tetrahydroisoxazolo[5,4-c]pyridin-3-ol (THIP/gaboxadol) or muscimol.

In another embodiment, the compound comprises a GABA uptake inhibitor. In one embodiment, the compound comprises tiagabine.

In another embodiment, the compound comprises a progesterone-based neurosteroid.

In a further embodiment of the invention, a method of treating psychiatric disorders related to the menstrual cycle comprises upregulating expression of δ subunit-containing GABA-A receptors.

In one embodiment, the upregulating step comprises administering a GABA-A receptor agonist.

In another embodiment, the upregulating step comprises administering a GABA uptake inhibitor.

In another embodiment, the upregulating step comprises administering a progesterone-based neurosteroid.

In still another embodiment of the invention, a method of treating PMS/PMDD or catamenial epilepsy comprises administering a non-toxic dose of a compound from the group consisting essentially of: specific agonists of δ subunit-containing GABA-A receptors, specific enhancers of GABA efficacy at δ subunit-containing GABA-A receptors, steroid anesthetics, barbiturates, and GABA uptake blockers.

In one embodiment, the compound comprises 4,5,6,7-tetrahydroisoxazolo[5,4-c]pyridin-3-ol (THIP/gaboxadol).

In another embodiment, the compound comprises muscimol.

In another embodiment, the compound comprises tiagabine.

In a further embodiment of the invention, a method of treating enhanced anxiety levels associated with the menstrual cycle comprises restoring tonic inhibition to normal levels.

In one embodiment, the restoring step comprises administering a non-toxic dose of a compound that targets δ subunit-containing GABA-A receptors.

In one embodiment, the compound comprises at least one compound from the group consisting essentially of: specific agonists of δ subunit-containing GABA-A receptors, specific enhancers of GABA efficacy at δ subunit-containing GABA-A receptors, steroid anesthetics, barbiturates, and GABA uptake blockers, and derivatives thereof.

In still another embodiment of the invention, a method of treating psychiatric disorders related to the menstrual cycle comprises potentiating δ subunit-containing GABA-A receptors during the luteal phase of the menstrual cycle.

In one embodiment, the restoring step comprises administering a non-toxic dose of a compound that targets δ subunit-containing GABA-A receptors.

In one embodiment, the compound comprises at least one compound from the group consisting essentially of: specific agonists of δ subunit-containing GABA-A receptors, specific enhancers of GABA efficacy at δ subunit-containing GABA-A receptors, steroid anesthetics, barbiturates, and GABA uptake blockers, and derivatives thereof.

Accordingly, an aspect of the invention is control of psychiatric disorders caused by cyclic changes in neurosteroid levels by targeting specific receptors in the brain that are related to control of neuronal excitability.

Another aspect of the invention is enhanced regulation of δ subunit-containing GABA-A receptors, which increases tonic inhibition and decreases both anxiety and seizure susceptibility.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWINGS

The invention will be more fully understood by reference to the following drawings which are for illustrative purposes only:

FIG. 1A depicts representative vaginal smears for mice in estrus and diestrus, respectively.

FIG. 1B is a graph of vaginal impedance versus time, showing that the estrus cycle is a regular seven-day cycle for mice.

FIG. 2 is a representation of subunit expression of GABA-A receptors.

FIG. 3A is a recording of tonic conductance in estrus and diestrus.

FIG. 3B is a graph of average tonic conductance in estrus and diestrus.

FIG. 3C is a recording of phasic currents over the estrous cycle.

### DETAILED DESCRIPTION OF THE INVENTION

The amino acid GABA is the major inhibitory transmitter in the mammalian brain. Alterations in its inhibitory function have been implicated in various psychiatric disorders, including depression, anxiety, bipolar disorders, schizophrenia, and stress-related disorders (Brambilla et al., 2003). Several drugs presently used to treat these disorders are known to act on specific membrane receptors for GABA. The GABA-A receptors (GABAARs) are members of the ligand-gated ion channel family and are activated by GABA. The GABAARs are pentameric hetero-oligomers assembled from seven different subunit classes with some subclasses having multiple members, as noted: α(1-6), β(1-3), γ(1-3), δ, ε, π, and ρ (Macdonald and Olsen, 1994; Sieghart and Sperk, 2002). In theory, a bewildering array of various heteropentameric combinations could result from the variety of subunits and their splice variants, but most GABAAR subtypes found in the brain form preferred assemblies (McKernan and Whiting, 1996; Sieghart and Sperk, 2002; Whiting, 2003). Studies in expression systems have revealed numerous differences between the physiological and pharmacological properties of GABAARs composed of different subunits (Hevers and Lüddens, 1998).

Neurons in the brains of mammals, including humans, show a large variety of GABAAR combinations, and some of the properties of neuronal receptors match those studied in expression systems (Hevers and Lüddens, 1998). Neurons communicate with each other through specialized structures called synapses, in which the presynaptic cell releases the neurotransmitter into the synaptic cleft from which it binds to the specialized receptors found on the adjacent membrane of the postsynaptic cell. It is now clear that GABAARs on neurons can be found either at synapses (synaptic receptors) or outside of synapses (extrasynaptic receptors). The extrasynaptic receptors appear to have a specific molecular composition and are activated by ambient levels of GABA present in the extracellular fluid in submicromolar concentrations (Mody, 2001). To date, we know of two subunit combinations that can be found extrasynaptically and are activated by the GABA present in the extracellular space, producing a steady current termed tonic inhibition. Tonic inhibition can be generated by δ subunit-containing GABAARs in cerebellar granule cells and hippocampal dentate gyrus granule cells and in various interneurons (Stell et al., 2003). In CA1 pyramidal cells, the tonic inhibition appears to be mediated by α5 subunit-containing receptors (Caraiscos et al., 2004). Tonic inhibition is a major force in dampening the excitability of central neurons. The current mediated by tonically activated GABA receptors is 4-5 fold larger than that produced by the activation of synaptic receptors (Nusser and Mody, 2002; Semyanov et al., 2004).

The δ subunit-containing GABAARs have recently emerged as having a specific pharmacology. They have an unusually high GABA affinity (Saxena and Macdonald, 1996; Brown et al., 2002) but low GABA efficacy (Bianchi and Macdonald, 2003). The low efficacy of GABA at these receptors allows for a large range of modulation of their activity by various compounds endogenous or exogenous to the brain. These compounds are chemicals or drugs that selectively activate the GABAARs containing δ subunits or selectively potentiate the effect of GABA at these receptors. The efficacy of GABA at these receptors is significantly enhanced by neuroactive steroids (Bianchi and Macdonald, 2003), ethanol (Wailner et al., 2003). Some specific agonists have a much higher efficacy than GABA. The class of drugs includes 4,5,6,7-tetrahydroisoxazolo[5,4-c]pyridin-3-ol (THIP or gaboxadol), muscimol, and barbiturates, or derivatives thereof.

The high sensitivity of δ subunit-containing GABA receptors to neurosteroids sets them apart from all other GABA receptors and makes these receptors of great physiological and pathological relevance. Neurosteroids are derived locally in the CNS from various steroid hormones synthesized in other parts of the body (Stoffel-Wagner, 2003). The precursors of neurosteroids include progesterone, the hormone secreted by the corpus luteum during the second phase of the menstrual cycle. Concentrations of the neurosteroid THDOC in the physiological range (10 nM) significantly potentiate the tonic conductance mediated by δ subunit-containing GABAARs in several classes of neurons (Stell et al., 2003) but have no effect on synaptic currents. Thus, a unique site of action for neurosteroids has been identified in the mammalian brain (Stell et al., 2003).

The specific distribution of δ subunit-containing neurosteroid-sensitive GABAARs found in the mammalian brain is regulated during the ovarian cycle. Accordingly, the level of tonic inhibition oscillates during various phases of the ovarian cycle, being highest when progesterone levels rise. Furthermore, anxiety levels of animals, as measured by their behavior in the elevated plus maze, are reduced when both expression of δ subunit-containing GABAARs and tonic inhibition are increased during late diestrus. Thus, a defective tonic inhibition mediated by neurosteroid-sensitive δ subunit-containing GABAARs might underlie the psychiatric disorders in women associated with the menstrual cycle. Accordingly, it should be possible to restore tonic inhibition to normal levels by the use and administration of a nontoxic dose of a compound that acts specifically on δ subunit-containing GABAARs. This compound should be a specific agonist of δ subunit-containing GABAARs or a specific enhancer of GABA efficacy at δ subunit-containing GABAARs. The class of drugs includes 4,5,6,7-tetrahydroisoxazolo[4,5-c]pyridin-3-ol (THIP; or gaboxadol), muscimol, barbiturates, steroid anesthetics (such as ganaxolone or alphaxolone), GABA uptake blockers (such as tiagabine or gabitril), or derivatives thereof.

### Experimental Results

The attached figures illustrate the scientific background and rationale for a method for treating certain psychiatric disorders associated with PMS/PMDD through the use and administration of a nontoxic dose of a specific agonist of δ subunit-containing GABAARs receptors or specific potentiators of GABA efficacy at δ subunit containing GABAARs found in the brain. We discovered specific changes in hippocampal GABAR subunits and tonic inhibition during the ovarian cycle in mice. During the phase of the cycle when high progesterone levels are present (late diestrus), the phase of the murine cycle that corresponds to the luteal phase in women, an elevated level of GABAR δ subunit expression was found in dentate gyrus granule cells and was accompanied by an enhanced tonic GABA conductance measured by whole-cell patch-clamp recordings.

### EXAMPLE 1

Example 1 relates to elevated expression of δ subunit containing GABAARs in the membranes of hippocampal dentate gyrus granule cells during the late diestrus phase of the estrous cycle in mice.

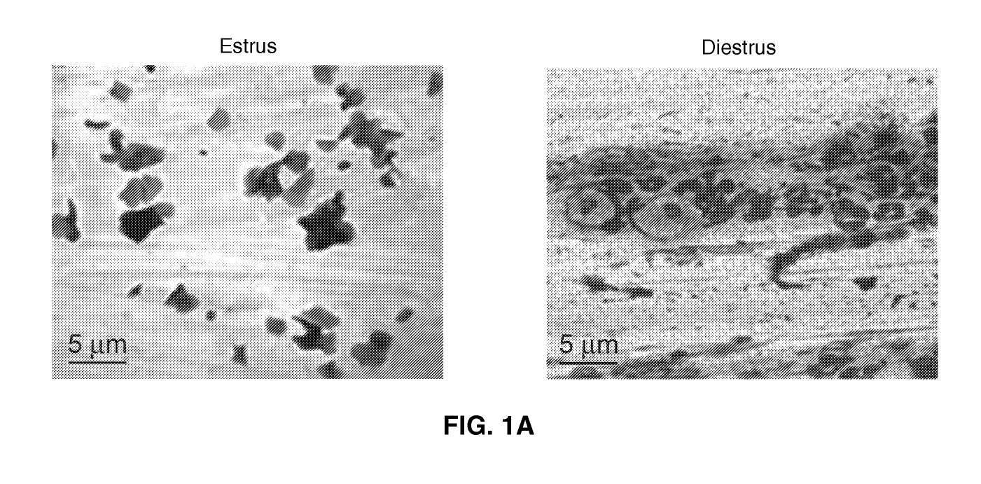

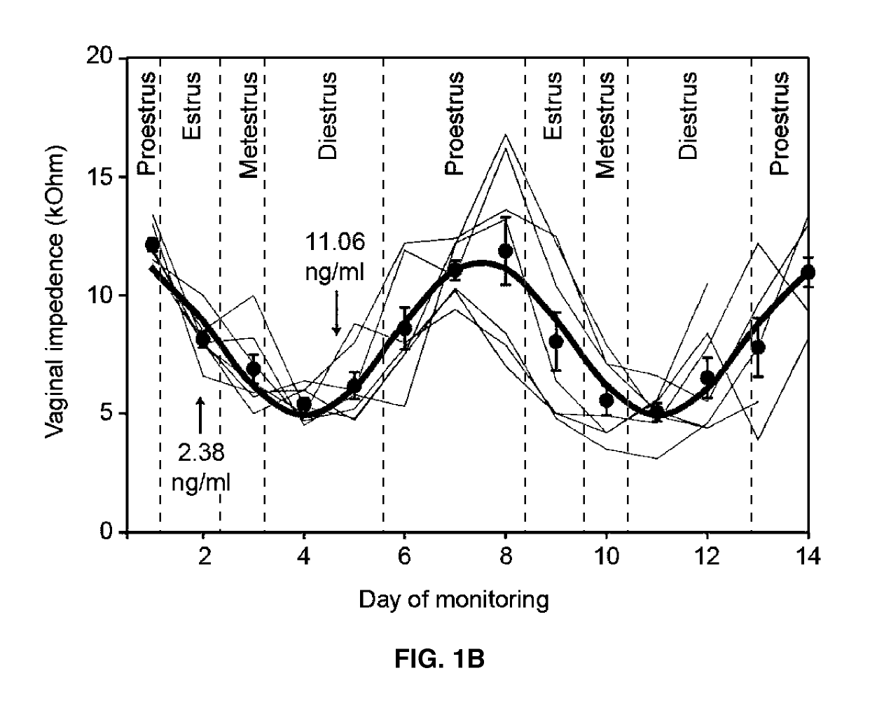

Discrimination between the different stages of the estrous cycle in mice was accomplished by analysis of the cellular profile of vaginal smears and measuring vaginal resistance. Referring to FIGS. 1A and 1B, the cellular profile of the estrus phase is characterized by the presence of irregularly shaped cornified cells, whereas the hallmark of diestrus is the presence of leukocytes and a sparse number of neurobasal cells. In addition to vaginal smears, vaginal impedance values were determined using an impedance monitor to detect vaginal resistance. The average vaginal impedance values over a fourteen-day period were fit with a sinusoidal wave function to determine the period to be exactly 7 days (FIG. 1B). These results indicate that the phase of the estrous cycle is consistently periodic and the different stages of the estrous cycle can be identified with confidence.

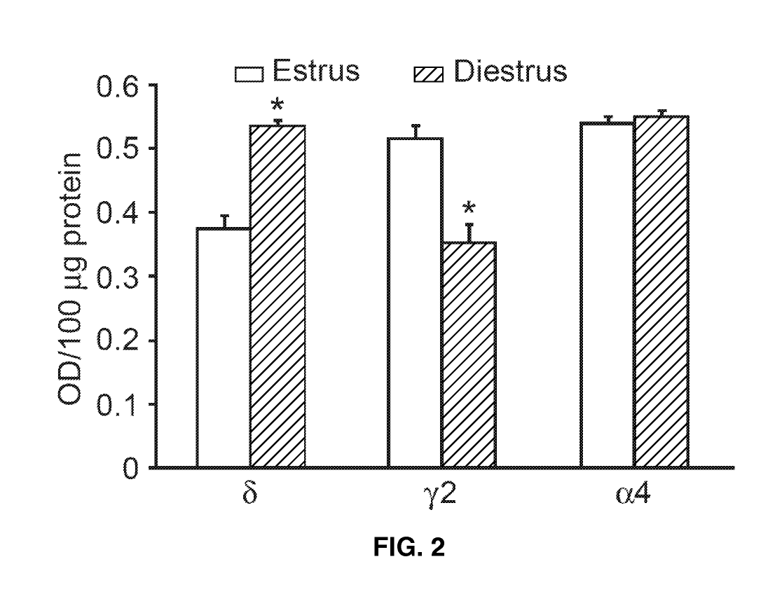

The expression of δ subunit-containing GABAARs was measure by immunoprecipitation using specific antibodies directed against these subunits. Immunoprecipitation was performed on total membrane protein and cytosolic protein isolated from the hippocampus of animals at different stages of the estrous cycle. See FIG. 2. Expression of the δ subunit in the membrane fraction is increased during diestrus compared to estrus and γ2 expression is concomitantly decreased. There is approximately a 15% increase in δ subunit expression and a 15% decrease in γ2 subunit expression in the membrane fraction of the hippocampus at diestrus. It is important to note that the increase in membrane δ subunit expression is not associated with a decrease in the cytosolic fraction of δ subunit expression, suggesting that trafficking of the δ subunit to the membrane coincides with increased production of the δ subunit in the cytosolic pool.

### EXAMPLE 2

Example 2 relates to changes in tonic inhibition secondary to the elevated expression of δ subunit containing GABAARs in dentate gyrus granule cells during the late diestrus phase of the estrous cycle in mice.

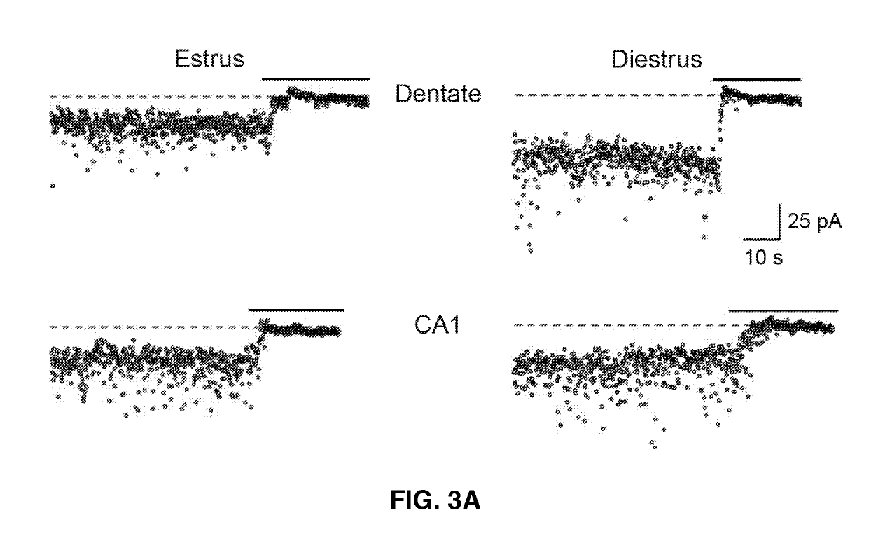

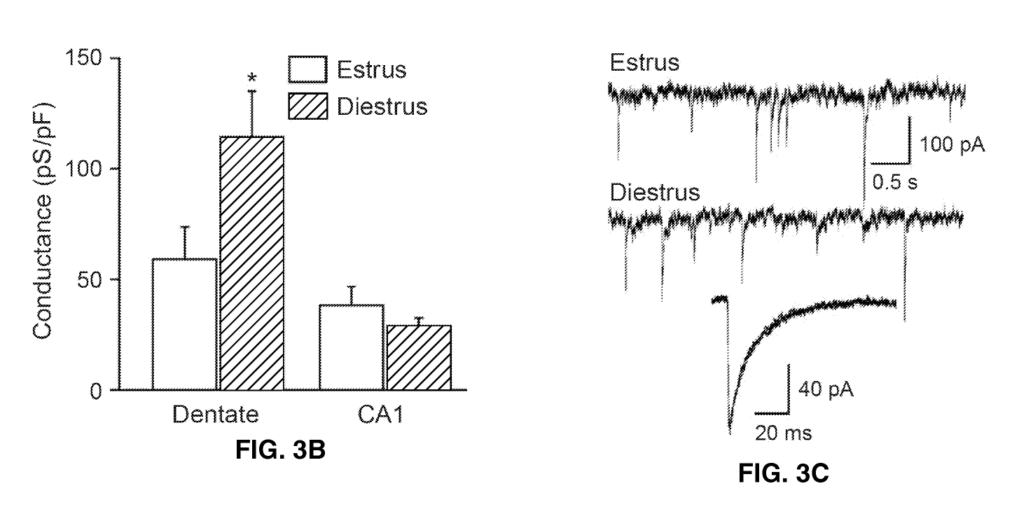

In order to investigate the functional changes resulting from alterations in GABAA receptor subunit expression over the estrous cycle, whole-cell patch recordings were performed in 350 μm coronal hippocampal slices. The GABAA receptor antagonist SR95531 (>100(mu)M) was used to reveal tonic inhibitory currents (Stell et al., 2003). Representative traces of tonic currents in DGGCs and CA1 pyramidal cells from estrus and diestrus mice are shown in FIG. 3A. There is a two-fold increase in the tonic conductance recorded from DGGCs from diestrus mice compared to estrus mice (FIG. 3B). The average tonic conductance in estrus mice is 59.16±17.87 pS/pF; whereas, the average tonic conductance in diestrus mice is 113.72±43.77 pS/pF. However, there is no significant change in the tonic conductance recorded in CA1 pyramidal cells, which is expected since δ subunit expression is low in the CA1 region of the hippocampus. The average tonic conductance recorded in CA1 pyramidal cells is 30.10±9.9 pS/pF and 29.37±3.4 pS/pF, in estrus and diestrus mice respectively. In addition, there are no significant changes in the kinetics of spontaneous inhibitory postsynaptic currents (sIPSCs) observed in either DGGCs or CA1 pyramidal cells over the course of the estrous cycle (FIG. 3C).

### EXAMPLE 3

Example 3 relates to lower levels of anxiety are associated with the enhanced tonic inhibition and elevated expression of δ subunit containing GABAARs during the late diestrus phase of the estrous cycle in mice.

Mice were tested for changes in anxiety levels by monitoring their behavior in the elevated plus maze, as noted in Table 1. Individual mice tested throughout the course of their estrous cycle entered the open arms less and spent less time in the open arms when they were in estrus compared to the late diestrus phase. The percentage of entries into the open arm (an indicator of low anxiety levels) is 5.68±1.11 for estrus mice and 9.69±2.29 for diestrus mice. Similarly, estrus mice spend 1.36±1.05 percent of time in the open arm compared to 3.80±0.32 for diestrus mice. In contrast, mice in diestrus entered the closed arms fewer times and spent less percentage of time in the closed arms compared to when these same mice were in estrus. The percentage of entries into the closed arm is 44.53±1.06 and 40.08±1.04 for estrus and diestrus mice, respectively. Estrus mice spend 93.71±0.69 percent of the time in the closed arm; whereas, diestrus mice only spend 86.90±1.68 percent of time in the closed arm. Interestingly, diestrus mice also spend significantly more time in the center of the elevated plus maze compared to estrus mice (9.26±1.83 compared to 4.91±0.57) (data not shown). In addition, diestrus mice also make significantly more total entries (37.47±3.64) compared to estrus mice (25±2.98) (data not shown). These data suggest that anxiety levels fluctuate related to the stage of the estrous cycle, such that mice in late diestrus exhibit decreased levels of anxiety compared to estrus.

Based on the findings presented above, the psychiatric symptoms of PMS/PMDD may arise from an insufficient potentiation of δ subunit-containing GABAARs during the luteal phase of the menstrual cycle. As dysfunctional GABA receptors are associated with mood disorders (Brambilla et al., 2003), it is postulated that women suffering from PMS/PMDD have a specific deficit in the increased functioning of δ subunit-containing GABAARs that should occur during the luteal phase. This deficit will result in the psychiatric symptoms associated with PMS/PMDD. The normal levels of δ subunit-containing GABAAR function should be restored by the use and administration of a nontoxic dose of a compound that acts specifically on δ subunit-containing GABAARs. Specifically, the administration of the following classes of drugs at the indicated ranges of dosages should be beneficial for the treatment of the psychiatric symptoms associated with PMS/PMDD:

1) Direct agonists with higher efficacy than GABA at δ subunit-containing GABAARs.

This class of drugs includes 4,5,6,7-tetrahydroisoxazolo[5,4-c]pyridin-3-ol (THIP/gaboxadol) and muscimol. THIP (gaboxadol, manufactured by Lundbeck/Merck) is presently in clinical trials for treating insomnia (i.e., a sleeping pill) at doses of 15-20 mg single treatment. Its dosage for the treatment of symptoms associated with PMS/PMDD should be between 1-15 mg. The dosage of muscimol will have to be determined.

2) Drugs that potentiate the efficacy of GABA at δ subunit-containing GABAARs.

This class of drugs includes low dose synthetic neurosteroids and barbiturates.

3) Drugs that increase ambient levels of GABA to act upon δ subunit-containing GABAARs.

This class of drugs includes inhibitors of GAT-1 mediated GABA uptake such as tiagabine (Gabitril, presently approved as an anticonvulsant), and inhibitors of metabolic GABA breakdown such a vigabatrin (Sabril, presently approved as an anticonvulsant).

The diagnostic application of this invention is the design of specific molecules or drugs that will bind with high affinity to δ subunit-containing GABAARs, and could be radioactively labeled to be used as a specific ligand for positron-emission tomography (PET) scans. Such compounds could then be used to screen for various abnormalities in the distribution, quantification or quantity of δ subunit-containing GABAARs during various phases of the menstrual cycle in control patients and women affected by PMS/PMDD.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

TABLE I
Effect of the Estrous Cycle on Anxiety
Evaluated Using the Elevated Plus Maze

| | Open Arms | | Closed Arms | |
|---|---|---|---|---|
| | Entries | % Time | Entries | % Time |
| Estrus | 5.68 ± 1.11 | 1.36 ± 1.05* | 44.53 ± 1.06 | 93.71 ± 0.69 |
| Diestrus | 9.69 ± 2.29* | 3.80 ± 0.32 | 40.08 ± 1.04* | 86.90 ± 1.68* |

*denotes statistical significance compared to mice in estrus

Individual mice were tested for 10 minutes on the elevated plus maze throughout the course of their estrous cycle. Activity was analyzed based upon the following parameters: time spent and entries into the open arms and closed arms. All values are expressed as percent of total. Significance was considered a p-value >0.05, determined using a paired t-test. n=6.

What is claimed is:

1. A method of regulating expression of δ subunit-containing GABA-A receptors, comprising:
administering a nontoxic dose of a compound targeting enhancement of neurosteroid-sensitive tonic GABAergic inhibition.

2. A method as recited in claim 1, wherein said compound comprises a GABA-A receptor agonist.

3. A method as recited in claim 2, wherein said compound comprises 4,5,6,7-tetrahydroisoxazolo[5,4-c]pyridin-3-ol (THIP/gaboxadol) or muscimol.

4. A method as recited in claim 1, wherein said compound comprises a GABA uptake inhibitor.

5. A method as recited in claim 4, wherein said compound comprises tiagabine.

6. A method as recited in claim 1, wherein said compound comprises a progesterone-based neurosteroid.

7. A method of treating psychiatric disorders related to the menstrual cycle, comprising:
upregulating expression of δ subunit-containing GABA-A receptors.

8. A method as recited in claim 7, wherein the upregulating step comprises administering a GABA-A receptor agonist.

9. A method as recited in claim 7, wherein the upregulating step comprises administering a GABA uptake inhibitor.

10. A method as recited in claim 7, wherein the upregulating step comprises administering a progesterone-based neurosteroid.

11. A method of treating PMS/PMDD or catamenial epilepsy, comprising:
administering a non-toxic dose of a compound from the group consisting essentially of: specific agonists of δ subunit-containing GABA-A receptors, specific enhancers of GABA efficacy at δ subunit-containing GABA-A receptors, steroid anesthetics, barbiturates, GABA uptake blockers, and derivatives thereof.

12. A method as recited in claim 11, wherein said compound comprises 4,5,6,7-tetrahydroisoxazolo[5,4-c]pyridin-3-ol (THIP/gaboxadol).

13. A method as recited in claim 11, wherein said compound comprises muscimol.

14. A method as recited in claim 11, wherein said compound comprises tiagabine.

15. A method of treating enhanced anxiety levels associated with the menstrual cycle, comprising:
restoring tonic inhibition to normal levels.

16. A method as recited in claim 15, wherein the restoring step comprises:
administering a non-toxic dose of a compound that targets δ subunit-containing GABA-A receptors.

17. A method as recited in claim 16, wherein said compound comprises at least one compound from the group consisting essentially of: specific agonists of δ subunit-containing GABA-A receptors, specific enhancers of GABA efficacy at δ subunit-containing GABA-A receptors, steroid anesthetics, barbiturates, and GABA uptake blockers, and derivatives thereof.

18. A method of treating psychiatric disorders related to the menstrual cycle, comprising:
potentiating δ subunit-containing GABA-A receptors during the luteal phase of the menstrual cycle.

19. A method as recited in claim 18, wherein said restoring step comprises:
administering a non-toxic dose of a compound that targets δ subunit-containing GABA-A receptors.

20. A method as recited in claim 19, wherein said compound comprises at least one compound from the group consisting essentially of: specific agonists of δ subunit-containing GABA-A receptors, specific enhancers of GABA efficacy at δ subunit-containing GABA-A receptors, steroid anesthetics, barbiturates, and GABA uptake blockers, and derivatives thereof.
